(19) United States
(12) Patent Application Publication
(10) Pub. No.: US 2006/0166235 A1
(43) Pub. Date: Jul. 27, 2006

(54) Methods, compositions, and kits for forming labeled polynucleotides

(75) Inventors: Lori K. Hennessy, San Mateo, CA (US); Mark R. Andersen, Carlsbad, CA (US)

Correspondence Address: MILA KASAN, PATENT DEPT.;APPLIED BIOSYSTEMS, 850 LINCOLN CENTRE DRIVE, FOSTER CITY, CA, 94404, US

(73) Assignee: Applera Corporation, Foster City, CA (US)

(21) Appl. No.: 11/303,765

(22) Filed: Dec. 15, 2005

Related U.S. Application Data
(60) Provisional application No. 60/640,127, filed on Dec. 29, 2004.

(51) Int. Cl. C12Q 1/68; C12P 19/34

(52) U.S. Cl. 435/6; 435/91.2

(57) ABSTRACT

The present teachings provide novel methods, compositions, and kits for forming multi-labeled polynucleotides. In some embodiments of the present teachings, multiplexed amplification reactions are performed with a plurality of primer pairs, wherein one primer in a given primer pair comprises a distinct label. Additional labeling of the resulting amplicons can be accomplished by using at least one bridge oligonucleotide to ligate a labeled tag oligonucleotide to each labeled extension product, thereby forming a plurality of multi-labeled polynucleotides. Detection of labels such as florophores and mobility modifiers in the plurality of multi-labeled polynucleotides can identify a sample. Such sample identification can be performed using a mobility dependent analysis technique such as capillary electrophoresis, and can applicable in the field of forensics.

### CROSS-REFERENCE TO RELATED APPLICATIONS

This application claims a priority benefit under 35 U.S.C. § 119(e) from U.S. Patent Application No. 60/640,127, filed Dec. 29, 2004, which is incorporated herein by reference.

### FIELD

The present teachings relate to methods, compositions, and kits for forming multi-labeled polynucleotides.

### INTRODUCTION

Numerous fields in molecular biology require the identification of target polynucleotide sequences. For example, multiplexed amplification of polymorphic genomic loci has been successfully used in human identification. Analysis of multiplexed amplification products using mobility dependent analysis techniques such as capillary electrophoresis can result in a collection of fragments that identify an organism. Multiplexed amplification reaction mixtures comprise a variety of molecular species. Approaches that reduce the complexity of amplified reaction mixtures are useful for simplifying data analysis.

### SUMMARY

In some embodiments, the present teachings provide a method of forming a multi-labeled polynucleotide comprising; hybridizing a labeled primer to a target polynucleotide; extending the labeled primer to form a labeled extension product; and, ligating a labeled tag oligonucleotide to the labeled extension product to form a multi-labeled polynucleotide.

In some embodiments, the present teachings provide a method of forming a multi-labeled polynucleotide comprising; providing a labeled primer and an unlabeled primer; amplifying a target polynucleotide with the labeled primer and the unlabeled primer in a PCR to form an amplicon, wherein the amplicon comprises a labeled extension product and an unlabeled extension product; ligating a labeled tag oligonucleotide to the labeled extension product to form a multi-labeled polynucleotide.

In some embodiments, the present teachings provide a method of forming at least two different multi-labeled polynucleotides comprising; providing a first primer pair specific for a first target polynucleotide, wherein the first primer pair comprises a first labeled primer and a first unlabeled primer; providing a second primer pair specific for a second target polynucleotide, wherein the second primer pair comprises a second labeled primer and a second unlabeled primer; amplifying the first target polynucleotide and the second target polynucleotide in a PCR to form a first amplicon and a second amplicon, wherein the first amplicon comprises a first labeled extension product and a first unlabeled extension product, and the second amplicon comprises a second labeled extension product and a second unlabeled extension product; ligating a first labeled tag oligonucleotide to the first labeled extension product ligating a second labeled tag oligonucleotide to the second labeled extension product; wherein the ligating comprises a first bridge oligonucleotide and a second bridge oligonucleotide, wherein the first labeled tag oligonucleotide and the first labeled extension product hybridize adjacent to one another on the first bridge oligonucleotide, and wherein the second labeled tag oligonucleotide and the second labeled extension product hybridize adjacent to one another on the second bridge oligonucleotide; and forming at least two different multi-labeled polynucleotides.

In some embodiments, the present teachings provide a kit comprising a primer pair, a bridge oligonucleotide, and a labeled tag oligonucleotide, wherein one primer in the primer pair comprises a label.

### BRIEF DESCRIPTION OF THE DRAWINGS

The skilled artisan will understand that the drawings, described below, are for illustration purposes only. The drawings are not intended to limit the scope of the present teachings in any way.

FIG. 1 depicts one embodiment for forming a multi-labeled polynucleotide according to some embodiments of the present teachings.

FIG. 2 depicts one embodiment for forming a multi-labeled polynucleotide according to some embodiments of the present teachings.

FIG. 3 depicts one embodiment for forming a multi-labeled polynucleotide wherein a 3′ adenine addition is queried.

### DETAILED DESCRIPTION OF EXEMPLARY EMBODIMENTS

Aspects of the present teachings may be further understood in light of the following exemplary embodiments, which should not be construed as limiting the scope of the present teachings in any way. The section headings used herein are for organizational purposes only and are not to be construed as limiting the described subject matter in any way. All literature and similar materials cited in this application, including but not limited to, patents, patent applications, articles, books, treatises, and inter-net web pages are expressly incorporated by reference in their entirety for any purpose. When definitions of terms in incorporated references appear to differ from the definitions provided in the present teachings, the definition provided in the present teachings shall control. It will be appreciated that there is an implied “about” prior to the temperatures, concentrations, times, etc discussed in the present teachings, such that slight and insubstantial deviations are within the scope of the present teachings herein. In this application, the use of the singular includes the plural unless specifically stated otherwise. Also, the use of “comprise”, “comprises”, “comprising”, “contain”, “contains”, “containing”, “include”, “includes”, and “including” are not intended to be limiting. It is to be understood that both the foregoing general description and the following detailed description are exemplary and explanatory only and are not restrictive of the invention.

Some Definitions

As used herein, the term “multi-labeled polynucleotide” refers to a polynucleotide that comprises at least two labels, typically, a label on each end. In some embodiments, a first label is introduced in first reaction such as a primer extension reaction, and a second label is added in a ligation reaction.

As used herein, the term “labeled primer” refers to a primer that can be extended, wherein the primer comprises a label.

As used herein, the term “target polynucleotide” refers to the substrate on which a primer hybridizes. In some embodiments, a labeled primer is hybridized on a target polynucleotide and extended to form a labeled extension product. The term “target polynucleotide” can refer to the target polynucleotide itself, as well as surrogates thereof, for example amplification products. In some embodiments, the target polynucleotide is a short DNA molecule derived from a degraded source, such as can be found in for example but not limited to forensics samples (see for example Butler, 2001, Forensic DNA Typing: Biology and Technology Behind STR Markers. The target polynucleotides of the present teachings can be derived from any of a number of sources, including without limitation, viruses, prokaryotes, eukaryotes, for example but not limited to plants, fungi, and animals. These sources may include, but are not limited to, whole blood, a tissue biopsy, lymph, bone marrow, amniotic fluid, hair, skin, semen, biowarfare agents, anal secretions, vaginal secretions, perspiration, saliva, buccal swabs, various environmental samples (for example, agricultural, water, and soil), research samples generally, purified samples generally, cultured cells, and lysed cells. It will be appreciated that target polynucleotides can be isolated from samples using any of a variety of procedures known in the art, for example the Applied Biosystems ABI Prism™ 6100 Nucleic Acid PrepStation, and the ABI Prism™ 6700 Automated Nucleic Acid Workstation, Boom et al., U.S. Pat. No. 5,234,809., the Flexigene kit (Qiagen), the Paragene kit (Gentra), and the mirVana RNA isolation kit (Ambion), etc. It will be appreciated that target polynucleotides can be cut or sheared prior to analysis, including the use of such procedures as mechanical force, sonication, restriction endonuclease cleavage, heat, or any method known in the art. In general, the target polynucleotides of the present teachings will be single stranded, though in some embodiments the target polynucleotide can be double stranded, and a single strand can result from denaturation. It will be appreciated that either strand of a double-stranded molecule can serve as the target polynucleotide.

The term “nucleotide base”, as used herein, refers to a substituted or unsubstituted aromatic ring or rings. In certain embodiments, the aromatic ring or rings contain at least one nitrogen atom. In certain embodiments, the nucleotide base is capable of forming Watson-Crick and/or Hoogsteen hydrogen bonds with an appropriately complementary nucleotide base. Exemplary nucleotide bases and analogs thereof include, but are not limited to, naturally occurring nucleotide bases adenine, guanine, cytosine, 6 methyl-cytosine, uracil, thymine, and analogs of the naturally occurring nucleotide bases, e.g., 7-deazaadenine, 7-deazaguanine, 7-deaza-8-azaguanine, 7-deaza-8-azaadenine, N6-Δ2-isopentenyladenine (6iA), N6-Δ2-isopentenyl-2-methylthioadenine (2ms6iA), N2-dimethylguanine (dmG), 7-methylguanine (7mG), inosine, nebularine, 2-aminopurine, 2-amino-6-chloropurine, 2,6-diaminopurine, hypoxanthine, pseudouridine, pseudocytosine, pseudoisocytosine, 5-propynylcytosine, isocytosine, isoguanine, 7-deazaguanine, 2-thiopyrimidine, 6-thioguanine, 4-thiothymine, 4-thiouracil, O6-methylguanine, N6-methyladenine, O4-methylthymine, 5,6-dihydrothymine, 5,6-dihydrouracil, pyrazolo[3,4-D]pyrimidines (see, e.g., U.S. Pat. Nos. 6,143,877 and 6,127,121 and PCT published application WO 01/38584), ethenoadenine, indoles such as nitroindole and 4-methylindole, and pyrroles such as nitropyrrole. Certain exemplary nucleotide bases can be found, e.g., in Fasman, 1989, Practical Handbook of Biochemistry and Molecular Biology, pp. 385-394, CRC Press, Boca Raton, Fla., and the references cited therein.

The term “nucleotide”, as used herein, refers to a compound comprising a nucleotide base linked to the C-1′ carbon of a sugar, such as ribose, arabinose, xylose, and pyranose, and sugar analogs thereof. The term nucleotide also encompasses nucleotide analogs. The sugar may be substituted or unsubstituted. Substituted ribose sugars include, but are not limited to, those riboses in which one or more of the carbon atoms, for example the 2′-carbon atom, is substituted with one or more of the same or different Cl, F, —R, —OR, —NR2 or halogen groups, where each R is independently H, C1-C6 alkyl or C5-C14 aryl. Exemplary riboses include, but are not limited to, 2′-(C1-C6)alkoxyribose, 2′-(C5-C14)aryloxyribose, 2′,3′-didehydroribose, 2′-deoxy-3′-haloribose, 2′-deoxy-3′-fluororibose, 2′-deoxy-3′-chlororibose, 2′-deoxy-3′-aminoribose, 2′-deoxy-3′-(C1-C6)alkylribose, 2′-deoxy-3′-(C1-C6)alkoxyribose and 2′-deoxy-3′-(C5-C14)aryloxyribose, ribose, 2′-deoxyribose, 2′,3′-dideoxyribose, 2′-haloribose, 2′-fluororibose, 2′-chlororibose, and 2′-alkylribose, e.g., 2′-O-methyl, 4′-anomeric nucleotides, 1′-anomeric nucleotides, 2′-4′- and 3′-4′-linked and other “locked” or “LNA”, bicyclic sugar modifications (see, e.g., PCT published application nos. WO 98/22489, WO 98/39352;, and WO 99/14226). Exemplary LNA sugar analogs within a polynucleotide include, but are not limited to, the structures:

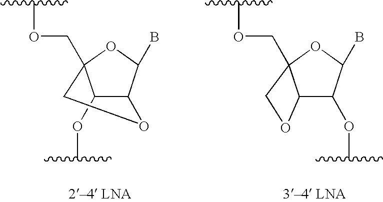

where B is any nucleotide base.

Modifications at the 2′- or 3′-position of ribose include, but are not limited to, hydrogen, hydroxy, methoxy, ethoxy, allyloxy, isopropoxy, butoxy, isobutoxy, methoxyethyl, alkoxy, phenoxy, azido, amino, alkylamino, fluoro, chloro and bromo. Nucleotides include, but are not limited to, the natural D optical isomer, as well as the L optical isomer forms (see, e.g., Garbesi (1993) Nucl. Acids Res. 21:4159-65; Fujimori (1990) J. Amer. Chem. Soc. 112:7435; Urata, (1993) Nucleic Acids Symposium Ser. No. 29:69-70). When the nucleotide base is purine, e.g. A or G, the ribose sugar is attached to the N9-position of the nucleotide base. When the nucleotide base is pyrimidine, e.g. C, T or U, the pentose sugar is attached to the N1-position of the nucleotide base, except for pseudouridines, in which the pentose sugar is attached to the C5 position of the uracil nucleotide base (see, e.g., Kornberg and Baker, (1992) DNA Replication, 2nd Ed., Freeman, San Francisco, Calif.).

One or more of the pentose carbons of a nucleotide may be substituted with a phosphate ester having the formula: 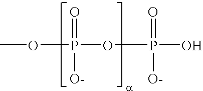
where a is an integer from 0 to 4. In certain embodiments, a is 2 and the phosphate ester is attached to the 3′- or 5′-carbon of the pentose. In certain embodiments, the nucleotides are those in which the nucleotide base is a purine, a 7-deazapurine, a pyrimidine, or an analog thereof. “Nucleotide 5′-triphosphate” refers to a nucleotide with a triphosphate ester group at the 5′ position, and are sometimes denoted as “NTP”, or “dNTP” and “ddNTP” to particularly point out the structural features of the ribose sugar. The triphosphate ester group may include sulfur substitutions for the various oxygens, e.g. -thio-nucleotide 5′-triphosphates. For a review of nucleotide chemistry, see: Shabarova, Z. and Bogdanov, A. Advanced Organic Chemistry of Nucleic Acids, VCH, New York, 1994.

The term “nucleotide analog”, as used herein, refers to embodiments in which the pentose sugar and/or the nucleotide base and/or one or more of the phosphate esters of a nucleotide may be replaced with its respective analog. In certain embodiments, exemplary pentose sugar analogs are those described above. In certain embodiments, the nucleotide analogs have a nucleotide base analog as described above. In certain embodiments, exemplary phosphate ester analogs include, but are not limited to, alkylphosphonates, methylphosphonates, phosphoramidates, phosphotriesters, phosphorothioates, phosphorodithioates, phosphoroselenoates, phosphorodiselenoates, phosphoroanilothioates, phosphoroanilidates, phosphoroamidates, boronophosphates, etc., and may Also included within the definition of “nucleotide analog” are nucleotide analog monomers which can be polymerized into polynucleotide analogs in which the DNA/RNA phosphate ester and/or sugar phosphate ester backbone is replaced with a different type of internucleotide linkage. Exemplary polynucleotide analogs include, but are not limited to, peptide nucleic acids, in which the sugar phosphate backbone of the polynucleotide is replaced by a peptide backbone. Also included are intercalating nucleic acids (INAs, as described in Christensen and Pedersen, 2002), and AEGIS bases (Eragen, U.S. Pat. No. 5,432,272).

As used herein, the terms “polynucleotide”, “oligonucleotide”, and “nucleic acid” are used interchangeably and mean single-stranded and double-stranded polymers of nucleotide monomers, including 2′-deoxyribonucleotides (DNA) and ribonucleotides (RNA) linked by internucleotide phosphodiester bond linkages, or internucleotide analogs, and associated counter ions, e.g., H+, NH4+, trialkylammonium, Mg2+, Na+ and the like. A nucleic acid may be composed entirely of deoxyribonucleotides, entirely of ribonucleotides, or chimeric mixtures thereof. The nucleotide monomer units may comprise any of the nucleotides described herein, including, but not limited to, naturally occuring nucleotides and nucleotide analogs. Nucleic acids typically range in size from a few monomeric units, e.g. 5-40 when they are sometimes referred to in the art as oligonucleotides, to several thousands of monomeric nucleotide units. Unless denoted otherwise, whenever a nucleic acid sequence is represented, it will be understood that the nucleotides are in 5′ to 3′ order from left to right and that “A” denotes deoxyadenosine or an analog thereof, “C” denotes deoxycytidine or an analog thereof, “G” denotes deoxyguanosine or an analog thereof, and “T” denotes thymidine or an analog thereof, unless otherwise noted.

Nucleic acids include, but are not limited to, genomic DNA, cDNA, hnRNA, mRNA, rRNA, tRNA, fragmented nucleic acid, nucleic acid obtained from subcellular organelles such as mitochondria or chloroplasts, and nucleic acid obtained from microorganisms or DNA or RNA viruses that may be present on or in a biological sample.

Nucleic acids may be composed of a single type of sugar moiety, e.g., as in the case of RNA and DNA, or mixtures of different sugar moieties, e.g., as in the case of RNA/DNA chimeras. In certain embodiments, nucleic acids are ribopolynucleotides and 2′-deoxyribopolynucleotides according to the structural formulae below:

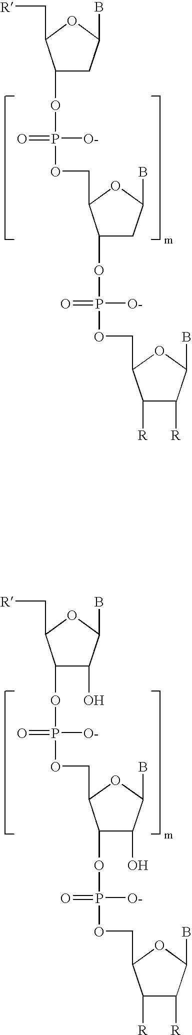

wherein each B is independently the base moiety of a nucleotide, e.g., a purine, a 7-deazapurine, a pyrimidine, or an analog nucleotide; each m defines the length of the respective nucleic acid and can range from zero to thousands, tens of thousands, or even more; each R is independently selected from the group comprising hydrogen, halogen, —R″, —OR″, and —NR″R″, where each R″ is independently (C1-C6)alkyl or (C5-C14)aryl, or two adjacent Rs are taken together to form a bond such that the ribose sugar is 2′,3′-didehydroribose; and each R′ is independently hydroxyl or

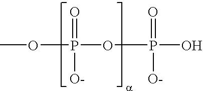

where a is zero, one or two.

In certain embodiments of the ribopolynucleotides and 2′-deoxyribopolynucleotides illustrated above, the nucleotide bases B are covalently attached to the C1′ carbon of the sugar moiety as previously described.

The terms “nucleic acid”, “polynucleotide”, and “oligonucleotide” may also include nucleic acid analogs, polynucleotide analogs, and oligonucleotide analogs. The terms “nucleic acid analog”, “polynucleotide analog” and “oligonucleotide analog” are used interchangeably and, as used herein, refer to a nucleic acid that contains at least one nucleotide analog and/or at least one phosphate ester analog and/or at least one pentose sugar analog. Also included within the definition of nucleic acid analogs are nucleic acids in which the phosphate ester and/or sugar phosphate ester linkages are replaced with other types of linkages, such as N-(2-aminoethyl)-glycine amides and other amides (see, e.g., Nielsen et al., 1991, Science 254: 1497-1500; WO 92/20702; U.S. Pat. No. 5,719,262; U.S. Pat. No. 5,698,685;); morpholinos (see, e.g., U.S. Pat. No. 5,698,685; U.S. Pat. No. 5,378,841; U.S. Pat. No. 5,185,144); carbamates (see, e.g., Stirchak & Summerton, 1987, J. Org. Chem. 52: 4202); methylene(methylimino) (see, e.g., Vasseur et al., 1992, J. Am. Chem. Soc. 114: 4006); 3′-thioformacetals (see, e.g., Jones et al., 1993, J. Org. Chem. 58: 2983); sulfamates (see, e.g., U.S. Pat. No. 5,470,967); 2-aminoethylglycine, commonly referred to as PNA (see, e.g., Buchardt, WO92/20702; Nielsen (1991) Science 254:1497-1500); and others (see, e.g., U.S. Pat. No. 5,817,781; Frier & Altman, 1997, Nucl. Acids Res. 25:4429 and the references cited therein). Phosphate ester analogs include, but are not limited to, (i) C1C4 alkylphosphonate, e.g. methylphosphonate; (ii) phosphoramidate; (iii) C1C6 alkyl-phosphotriester; (iv) phosphorothioate; and (v) phosphorodithioate.

As used herein, the term “labeled extension product” refers to the result of an extension reaction, wherein a labeled primer is incorporated into a nucleic acid strand. In some embodiments, a labeled extension product results from the hybridization and extension of a labeled primer to a target polynucleotide.

As used herein, the term “labeled tag oligonucleotide” refers to an oligonucleotide comprising a label, which can be ligated to an extension product. In some embodiments of the present teachings, the labeled tag oligonucleotide and a labeled extension product can be hybridized adjacently on a bridge oligonucleotide and subsequently ligated together.

As used herein, the term “unlabeled primer” refers to a primer that is present in a reaction with a labeled primer, and which is incorporated into an amplicon resulting from amplification of a target polynucleotide, such as for example in a PCR.

As used herein, the term “amplifying” refers to any means by which at least a part of a target polynucleotide, target polynucleotide surrogate, or combinations thereof, is reproduced, typically in a template-dependent manner, including without limitation, a broad range of techniques for amplifying nucleic acid sequences, either linearly or exponentially. Exemplary means for performing an amplifying step include ligase chain reaction (LCR), ligase detection reaction (LDR), ligation followed by Q-replicase amplification, PCR, primer extension, strand displacement amplification (SDA), hyperbranched strand displacement amplification, multiple displacement amplification (MDA), nucleic acid strand-based amplification (NASBA), two-step multiplexed amplifications, rolling circle amplification (RCA) and the like, including multiplex versions or combinations thereof, for example but not limited to, OLA/PCR, PCR/OLA, LDR/PCR, PCR/PCR/LDR, PCR/LDR, LCR/PCR, PCR/LCR (also known as combined chain reaction—CCR), and the like. Descriptions of such techniques can be found in, among other places, Sambrook et al. Molecular Cloning, 3^{rd} Edition,; Ausbel et al.; PCR Primer: A Laboratory Manual, Diffenbach, Ed., Cold Spring Harbor Press (1995); The Electronic Protocol Book, Chang Bioscience (2002), Msuih et al., J. Clin. Micro. 34:501-07 (1996); The Nucleic Acid Protocols Handbook, R. Rapley, ed., Humana Press, Totowa, N.J. (2002); Abramson et al., Curr Opin Biotechnol. 1993 February;4(1):41-7, U.S. Pat. No. 6,027,998; U.S. Pat. No. 6,605,451, Barany et al., PCT Publication No. WO 97/31256; Wenz et al., PCT Publication No. WO 01/92579; Day et al., Genomics, 29(1): 152-162 (1995), Ehrlich et al., Science 252:1643-50 (1991); Innis et al., PCR Protocols: A Guide to Methods and Applications, Academic Press (1990); Favis et al., Nature Biotechnology 18:561-64 (2000); and Rabenau et al., Infection 28:97-102 (2000); Belgrader, Barany, and Lubin, Development of a Multiplex Ligation Detection Reaction DNA Typing Assay, Sixth International Symposium on Human Identification, 1995 (available on the world wide web at: promega.com/geneticidproc/ussymp6proc/blegrad.html); LCR Kit Instruction Manual, Cat. #200520, Rev. #050002, Stratagene, 2002; Barany, Proc. Natl. Acad. Sci. USA 88:188-93 (1991); Bi and Sambrook, Nucl. Acids Res. 25:2924-2951 (1997); Zirvi et al., Nucl. Acid Res. 27:e40i-viii (1999); Dean et al., Proc Natl Acad Sci USA 99:5261-66 (2002); Barany and Gelfand, Gene 109:1-11 (1991); Walker et al., Nucl. Acid Res. 20:1691-96 (1992); Polstra et al., BMC Inf. Dis. 2:18-(2002); Lage et al., Genome Res. 2003 February;13(2):294-307, and Landegren et al., Science 241:1077-80 (1988), Demidov, V., Expert Rev Mol Diagn. 2002 November;2(6):542-8., Cook et al., J Microbiol Methods. 2003 May;53(2):165-74, Schweitzer et al., Curr Opin Biotechnol. 2001 February;12(1):21-7, U.S. Pat. No. 5,830,711, U.S. Pat. No. 6,027,889, U.S. Pat. No. 5,686,243, Published P.C.T. Application WO0056927A3, and Published P.C.T. Application WO9803673A1. In some embodiments, newly-formed nucleic acid duplexes are not initially denatured, but are used in their double-stranded form in one or more subsequent steps. In some embodiments of the present teachings, unconventional nucleotide bases can be introduced into the amplification reaction products and the products treated by enzymatic (e.g., glycosylases) and/or physical-chemical means in order to render the product incapable of acting as a template for subsequent amplifications. In some embodiments, uracil can be included as a nucleobase in the reaction mixture, thereby allowing for subsequent reactions to decontaminate carryover of previous uracil-containing products by the use of uracil-N-glycosylase (see for example Published P.C.T. Application WO9201814A2, U.S. Pat. No. 5,536,649, and U.S. Provisional Application 60/584,682 to Andersen et al., wherein UNG decontamination and phosphorylation are performed in the same reaction mixture, which further comprises a heat-activatable ligase.). In some embodiments of the present teachings, any of a variety of techniques can be employed prior to amplification in order to facilitate amplification success, as described for example in Radstrom et al., Mol Biotechnol. 2004 February;26(2):133-46. In some embodiments, amplification can be achieved in a self-contained integrated approach comprising sample preparation and detection, as described for example in U.S. Pat. Nos. 6,153,425 and 6,649,378. Reversibly modified enzymes, for example but not limited to those described in U.S. Pat. No. 5,773,258, are also within the scope of the disclosed teachings. Those in the art will understand that any protein with the desired enzymatic activity can be used in the disclosed methods and kits. Descriptions of DNA polymerases, including reverse transcriptases, uracil N-glycosylase, and the like, can be found in, among other places, Twyman, Advanced Molecular Biology, BIOS Scientific Publishers, 1999; Enzyme Resource Guide, rev. 092298, Promega, 1998; Sambrook and Russell; Sambrook et al.; Lehninger; PCR: The Basics; and Ausbel et al.

As used herein “ligation” comprises any enzymatic or non-enzymatic means wherein an inter-nucleotide linkage is formed between the opposing ends of nucleic acid sequences that are adjacently hybridized to a template. In some embodiments, ligation also comprises at least one gap-filling procedure, wherein the ends of the two probes are not adjacently hybridized initially but the 3′-end of the upstream probe is extended by one or more nucleotide until it is adjacent to the 5′-end of the downstream probe, typically by a polymerase (see, e.g., U.S. Pat. No. 6,004,826). The internucleotide linkage can include, but is not limited to, phosphodiester bond formation. Such bond formation can include, without limitation, those created enzymatically by at least one DNA ligase or at least one RNA ligase, for example but not limited to, T4 DNA ligase, T4 RNA ligase, Thermus thermophilus (Tth) ligase, Thermus aquaticus (Taq) DNA ligase, Thermus scotoductus (Tsc) ligase, TS2126 (a thermophilic phage that infects Tsc) RNA ligase, Archaeoglobus flugidus (Afu) ligase, Pyrococcus furiosus (Pfu) ligase, or the like, including but not limited to reversibly inactivated ligases (see, e.g., U.S. Pat. No. 5,773,258), and enzymatically active mutants and variants thereof. Other internucleotide linkages include, without limitation, covalent bond formation between appropriate reactive groups such as between an α-haloacyl group and a phosphothioate group to form a thiophosphorylacetylamino group, a phosphorothioate a tosylate or iodide group to form a 5′-phosphorothioester, and pyrophosphate linkages. Chemical ligation can, under appropriate conditions, occur spontaneously such as by autoligation. Alternatively, “activating” or reducing agents can be used. Examples of activating and reducing agents include, without limitation, carbodiimide, cyanogen bromide (BrCN), imidazole, 1-methylimidazole/carbodiimide/cystamine, N-cyanoimidazole, dithiothreitol (DTT) and ultraviolet light, such as used for photoligation. In some embodiments ligation can provide amplification in and of itself, as well as provide for an initial amplification followed by a subsequent amplification. In some embodiments of the present teachings, unconventional nucleotide bases can be introduced into the ligation probes and the resulting products treated by enzymatic (e.g., glycosylases) and/or physical-chemical means in order to render the product incapable of acting as a template for subsequent downstream reactions such as amplification. In some embodiments, uracil can be included as a nucleobase in the ligation reaction mixture, thereby allowing for subsequent reactions to decontaminate carryover of previous uracil-containing products by the use of uracil-N-glycosylase. Various approaches to decontamination using glycosylases and the like can be found for example in Published P.C.T. Application WO9201814A2). Methods for removing unhybridized and/or unligated probes following a ligation reaction are known in the art, and are further discussed supra. Such procedures include nuclease-mediated approaches, dilution, size exclusion approaches, affinity moiety procedures, (see for example U.S. Provisional Application 60/517,470, U.S. Provisional Application 60/477,614, and P.C.T. Application 2003/37227), affinity-moiety procedures involving immobilization of target polynucleotides (see for example Published P.C.T. Application WO 03/006677A2). The present teachings further contemplate approaches for removing contamination products using uracil glycosylases in concert with phosphorylation reactions and/or ligation reaction reactions, as described for example in Andersen et al., U.S. Provisional Application 60/584,682.

As used herein, the term “ligase” and “ligation agent” are used interchangeably and refer to any number of enzymatic or non-enzymatic reagents capable of joining an extension product to a labeled tag oligonucleotide. For example, ligase is an enzymatic ligation reagent that, under appropriate conditions, forms phosphodiester bonds between the 3′-OH and the 5′-phosphate of adjacent nucleotides in DNA molecules, RNA molecules, or hybrids. Temperature sensitive ligases, include, but are not limited to, bacteriophage T4 ligase and E. coli ligase. Thermostable ligases include, but are not limited to, Afu ligase, Taq ligase, Tfl ligase, Tth ligase, Tth HB8 ligase, Thermus species AK16D ligase and Pfu ligase (see for example Published P.C.T. Application WO00/26381, Wu et al., Gene, 76(2):245-254, (1989), Luo et al., Nucleic Acids Research, 24(15): 3071-3078 (1996). The skilled artisan will appreciate that any number of thermostable ligases, including DNA ligases and RNA ligases, can be obtained from thermophilic or hyperthermophilic organisms, for example, certain species of eubacteria and archaea; and that such ligases can be employed in the disclosed methods and kits. Chemical ligation agents include, without limitation, activating, condensing, and reducing agents, such as carbodiimide, cyanogen bromide (BrCN), N-cyanoimidazole, imidazole, 1-methylimidazole/carbodiimide/cystamine, dithiothreitol (DTT) and ultraviolet light. Autoligation, i.e., spontaneous ligation in the absence of a ligating agent, is also within the scope of the teachings herein. Detailed protocols for chemical ligation methods and descriptions of appropriate reactive groups can be found in, among other places, Xu et al., Nucleic Acid Res., 27:875-81 (1999); Gryaznov and Letsinger, Nucleic Acid Res. 21:1403-08 (1993); Gryaznov et al., Nucleic Acid Res. 22:2366-69 (1994); Kanaya and Yanagawa, Biochemistry 25:7423-30 (1986); Luebke and Dervan, Nucleic Acids Res. 20:3005-09 (1992); Sievers and von Kiedrowski, Nature 369:221-24 (1994); Liu and Taylor, Nucleic Acids Res. 26:3300-04 (1999); Wang and Kool, Nucleic Acids Res. 22:2326-33 (1994); Purmal et al., Nucleic Acids Res. 20:3713-19 (1992); Ashley and Kushlan, Biochemistry 30:2927-33 (1991); Chu and Orgel, Nucleic Acids Res. 16:3671-91 (1988); Sokolova et al., FEBS Letters 232:153-55 (1988); Naylor and Gilham, Biochemistry 5:2722-28 (1966); and U.S. Pat. No. 5,476,930. Photoligation using light of an appropriate wavelength as a ligation agent is also within the scope of the teachings. In some embodiments, photoligation comprises oligonucleotides comprising nucleotide analogs, including but not limited to, 4-thiothymidine (s⁴T), 5-vinyluracil and its derivatives, or combinations thereof. In some embodiments, the ligation agent comprises: (a) light in the UV-A range (about 320 nm to about 400 nm), the UV-B range (about 290 nm to about 320 nm), or combinations thereof, (b) light with a wavelength between about 300 nm and about 375 nm, (c) light with a wavelength of about 360 nm to about 370 nm; (d) light with a wavelength of about 364 nm to about 368 nm, or (e) light with a wavelength of about 366 nm. In some embodiments, photoligation is reversible. Descriptions of photoligation can be found in, among other places, Fujimoto et al., Nucl. Acid Symp. Ser. 42:39-40 (1999); Fujimoto et al., Nucl. Acid Res. Suppl. 1:185-86 (2001); Fujimoto et al., Nucl. Acid Suppl., 2:155-56 (2002); Liu and Taylor, Nucl. Acid Res. 26:3300-04 (1998) and on the world wide web at: sbchem.kyoto-u.ac.jp/saito-lab.

As used herein, the term “microsatellite” refers to a genetic locus comprising a short (e.g., 1-6 nucleotide), tandemly repeated sequence motif. Microsatellites are also known as short tandem repeats (STRs) in the art. They are widely dispersed and abundant in the eukaryotic genome, and are often highly polymorphic due to variation in the number of repeat units. This polymorphism renders microsatellites attractive DNA markers for genetic mapping, medical diagnostics and forensic investigation.

As used herein, the term “bridge oligonucleotide” refers to an oligonucleotide that can provide a substrate for the hybridization and subsequent ligation of an extension product and a labeled tag oligonucleotide. In some embodiments, a labeled extension product is hybridized adjacent to a labeled tag oligonucleotide on a bridge oligonucleotide, and their ligation results in a multi-labeled polynucleotide.

As used herein, the term “template-independent adenine” refers to the result of an enzymatic reaction in which nucleotide triphosphates, in particular an adenine, is covalently attached to the 3′ terminus of an polynucleotide in a template independent manner, known sometimes in the art as terminal transferase activity.

As used herein, the terms “annealing” and “hybridization” are used interchangeably and mean the complementary base-pairing interaction of one nucleic acid with another nucleic acid that results in formation of a duplex, triplex, or other higher-ordered structure. In some embodiments, the primary interaction is base specific, e.g., A/T and G/C, by Watson/Crick and Hoogsteen-type hydrogen bonding. In some embodiments, base-stacking and hydrophobic interactions may also contribute to duplex stability. Conditions for hybridizing nucleic acid sequences to complementary and substantially complementary nucleic sequences are well known, e.g., as described in Nucleic Acid Hybridization, A Practical Approach, B. Hames and S. Higgins, eds., IRL Press, Washington, D.C. (1985) and J. Wetmur and N. Davidson, Mol. Biol. 31:349 et seq. (1968). In general, whether such annealing takes place is influenced by, among other things, the length of the sequences, the pH, the temperature, the presence of mono- and divalent cations, the proportion of G and C nucleotides in the hybridizing region, the viscosity of the medium, and the presence of denaturants. Such variables influence the time required for hybridization. Thus, the preferred annealing conditions will depend upon the particular application. Such conditions, however, can be routinely determined by the person of ordinary skill in the art without undue experimentation. Further, in general nucleic acids of the present teachings are designed to be complementary to their corresponding sequence, such that hybridization occurs. It will be appreciated, however, that this complementarity need not be perfect; there can be any number of base pair mismatches that will interfere with hybridization between the corresponding sequences of the present teachings. However, if the number of base pair mismatches is so great that no hybridization can occur under even the least stringent of hybridization conditions, the sequences are not a complementary. Thus, by “substantially complementary” herein is meant that the sequences are sufficiently complementary to hybridize under the selected reaction conditions.

As used herein, the term “label” refers to any moiety that, when attached to a nucleotide or polynucleotide, renders such nucleotide or polynucleotide detectable using known detection methods. Labels may be direct labels which themselves are detectable or indirect labels which are detectable in combination with other agents. Exemplary direct labels include but are not limited to fluorophores, chromophores, radioisotopes (e.g., ³²P, ³⁵ S, ³H), spin-labels, Quantum Dots, chemiluminescent labels, and the like. Exemplary indirect labels include enzymes that catalyze a signal-producing event, and ligands such as an antigen or biotin that can bind specifically with high affinity to a detectable anti-ligand, such as a labeled antibody or avidin. Many comprehensive reviews of methodologies for labeling DNA provide guidance applicable to the present invention. Such reviews include Matthews et al. (1988); Haugland (1992), Keller and Manak (1993); Eckstein (1991); Kricka (1992), and the like. Also see U.S. Pat. Nos. 5,654,419, 5,707,804, 5,688,648, 6,028,190, 5,869,255, 6,177,247, 6,544,744, 5,728,528, and U.S. patent application Ser. No. 10/288,104. Labels can further refer to “mobility modifiers.”

As used herein, the term “mobility modifier” refers to a polymer chain that imparts to an oligonucleotide an electrophoretic mobility in a sieving or non-sieving matrix that is distinctive relative to the electrophoretic mobilities of the other polymer chains in a mixture. Typically, a mobility modifier changes the charge/translational frictional drag when hybridized or bound to the element; or imparts a distinctive mobility, for example but not limited to, a distinctive elution characteristic in a chromatographic separation medium or a distinctive electrophoretic mobility in a sieving matrix or non-sieving matrix, when hybridized or bound to the corresponding element; or both (see, e.g., U.S. Pat. Nos. 5,470,705 and 5,514,543). For various examples of mobilitity modifiers see for example U.S. Pat. Nos. 6,395,486, 6,358,385, 6,355,709, 5,916,426, 5,807,682, 5,777,096, 5,703,222, 5,556,7292, 5,567,292, 5,552,028, 5,470,705, and Barbier et al., Current Opinion in Biotechnology, 2003, 14:1:51-57. In some embodiments, at least one mobility modifier comprises at least one nucleotide polymer chain, including without limitation, at least one oligonucleotide polymer chain, at least one polynucleotide polymer chain, or both at least one oligonucleotide polymer chain and at least one polynucleotide polymer chain (see for example Published P.C.T. application WO9615271A1, as well as product literature for Keygene SNPWave™ for some examples of using known numbers of nucleotides to confer mobility to ligation products). In some embodiments, at least one mobility modifier comprises at least one non-nucleotide polymer chain. Exemplary non-nucleotide polymer chains include, without limitation, peptides, polypeptides, polyethylene oxide (PEO), or the like. In some embodiments, at least one polymer chain comprises at least one substantially uncharged, water-soluble chain, such as a chain composed of PEO units; a polypeptide chain; or combinations thereof. The polymer chain can comprise a homopolymer, a random copolymer, a block copolymer, or combinations thereof. Furthermore, the polymer chain can have a linear architecture, a comb architecture, a branched architecture, a dendritic architecture (e.g., polymers containing polyamidoamine branched polymers, Polysciences, Inc. Warrington, Pa.), or combinations thereof. In some embodiments, at least one polymer chain is hydrophilic, or at least sufficiently hydrophilic when hybridized or bound to an element to ensure that the element-mobility modifier is readily soluble in aqueous medium. Where the mobility-dependent analysis technique is electrophoresis, in some embodiments, the polymer chains are uncharged or have a charge/subunit density that is substantially less than that of its corresponding element. The synthesis of polymer chains useful as mobility modifiers will depend, at least in part, on the nature of the polymer. Methods for preparing suitable polymers generally follow well-known polymer subunit synthesis methods. These methods, which involve coupling of defined-size, multi-subunit polymer units to one another, either directly or through charged or uncharged linking groups, are generally applicable to a wide variety of polymers, such as polyethylene oxide, polyglycolic acid, polylactic acid, polyurethane polymers, polypeptides, oligosaccharides, and nucleotide polymers. Such methods of polymer unit coupling are also suitable for synthesizing selected-length copolymers, e.g., copolymers of polyethylene oxide units alternating with polypropylene units. Polypeptides of selected lengths and amino acid composition, either homopolymer or mixed polymer, can be synthesized by standard solid-phase methods (e.g., Int. J. Peptide Protein Res., 35: 161-214 (1990)). One method for preparing PEO polymer chains having a selected number of hexaethylene oxide (HEO) units, an HEO unit is protected at one end with dimethoxytrityl (DMT), and activated at its other end with methane sulfonate. The activated HEO is then reacted with a second DMT-protected HEO group to form a DMT-protected HEO dimer. This unit-addition is then carried out successively until a desired PEO chain length is achieved (e.g., U.S. Pat. No. 4,914,210; see also, U.S. Pat. No. 5,777,096).

As used herein, the term “fluorophore” refers to a label that comprises a resonance-delocalized system or aromatic ring system that absorbs light at a first wavelength and emits fluorescent light at a second wavelength in response to the absorption event. A wide variety of such dye molecules are known in the art. For example, fluorescent dyes can be selected from any of a variety of classes of fluorescent compounds, such as xanthenes, rhodamines, fluoresceins, cyanines, phthalocyanines, squaraines, and bodipy dyes. In some embodiments, the dye comprises a xanthene-type dye, which contains a fused three-ring system of the form: 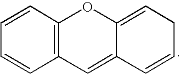
This parent xanthene ring may be unsubstituted (i.e., all substituents are H) or can be substituted with one or more of a variety of the same or different substituents, such as described below. In some embodiments, the dye contains a parent xanthene ring having the general structure:

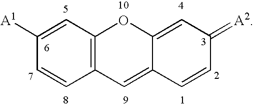

In the parent xanthene ring depicted above, A¹ is OH or NH₂ and A² is O or NH₂⁺. When A¹ is OH and A² is O, the parent xanthene ring is a fluorescein-type xanthene ring. When A¹ is NH₂ and A² is NH₂⁺, the parent xanthene ring is a rhodamine-type xanthene ring. When A¹ is NH₂ and A² is O, the parent xanthene ring is a rhodol-type xanthene ring. In the parent xanthene ring depicted above, one or both nitrogens of A¹ and A² (when present) and/or one or more of the carbon atoms at positions C1, C2, C4, C5, C7, C8 and C9 can be independently substituted with a wide variety of the same or different substituents. In some embodiments, typical substituents can include, but are not limited to, —X, —R, —OR, —SR, —NRR, perhalo (C₁-C₆)alkyl, —CX₃, —CF₃, —CN, —OCN, —SCN, —NCO, —NCS, —NO, —NO₂, —N₃, —S(O)₂O⁻, —S(O)₂OH, —S(O)₂R, —C(O)R, —C(O)X, —C(S)R, —C(S)X, —C(O)OR, —C(O)O⁻, —C(S)OR, —C(O)SR, —C(S)SR, —C(O)NRR, —C(S)NRR and —C(NR)NRR, where each X is independently a halogen (preferably —F or Cl) and each R is independently hydrogen, (C₁-C₆)alkyl, (C₁-C₆)alkanyl, (C₁-C₆)alkenyl, (C₁-C₆)alkynyl, (C₅-C₂₀)aryl, (C₆-C₂₆)arylalkyl, (C₅-C₂₀) arylaryl, heteroaryl, 6-26 membered heteroarylalkyl 5-20 membered heteroaryl-heteroaryl, carboxyl, acetyl, sulfonyl, sulfinyl, sulfone, phosphate, or phosphonate. Moreover, the C1 and C2 substituents and/or the C7 and C8 substituents can be taken together to form substituted or unsubstituted buta[1,3]dieno or (C₅-C₂₀)aryleno bridges. Generally, substituents that do not tend to quench the fluorescence of the parent xanthene ring are preferred, but in some embodiments quenching substituents may be desirable. Substituents that tend to quench fluorescence of parent xanthene rings are electron-withdrawing groups, such as —NO₂, —Br, and —I. In some embodiments, C9 is unsubstituted. In some embodiments, C9 is substituted with a phenyl group. In some embodiments, C9 is substituted with a substituent other than phenyl. When A¹ is NH₂ and/or A² is NH₂⁺, these nitrogens can be included in one or more bridges involving the same nitrogen atom or adjacent carbon atoms, e.g., (C₁-C₁₂)alkyldiyl, (C₁-C₁₂)alkyleno, 2-12 membered heteroalkyldiyl and/or 2-12 membered heteroalkyleno bridges. Any of the substituents on carbons C1, C2, C4, C5, C7, C8, C9 and/or nitrogen atoms at C3 and/or C6 (when present) can be further substituted with one or more of the same or different substituents, which are typically selected from —X, —R′, ═O, —OR′, —SR′, ═S, —NR′R′, ═NR′, —CX₃, —CN, —OCN, —SCN, —NCO, —NCS, —NO, —NO₂, ═N₂, —N₃, —NHOH, —S(O)₂O⁻, —S(O)₂OH, —S(O)₂R′, —P(O)(O⁻)₂, —P(O)(OH)₂, —C(O)R′, —C(O)X, —C(S)R′, —C(S)X, —C(O)OR′, —C(O)O⁻, —C(S)OR′, —C(O)SR′, —C(S)SR′, —C(O)NR′R′, —C(S)NR′R′ and —C(NR)NR′R′, where each X is independently a halogen (preferably —F or —Cl) and each R′ is independently hydrogen, (C₁-C₆)alkyl, 2-6 membered heteroalkyl, (C₅-C₁₄)aryl or heteroaryl, carboxyl, acetyl, sulfonyl, sulfinyl, sulfone, phosphate, or phosphonate.

Exemplary parent xanthene rings include, but are not limited to, rhodamine-type parent xanthene rings and fluorescein-type parent xanthene rings.

In one embodiment, the dye contains a rhodamine-type xanthene dye that includes the following ring system:

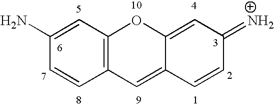

In the rhodamine-type xanthene ring depicted above, one or both nitrogens and/or one or more of the carbons at positions C1, C2, C4, C5, C7 or C8 can be independently substituted with a wide variety of the same or different substituents, as described above for the parent xanthene rings, for example. C9 may be substituted with hydrogen or other substituent, such as an orthocarboxyphenyl or ortho(sulfonic acid)phenyl group. Exemplary rhodamine-type xanthene dyes can include, but are not limited to, the xanthene rings of the rhodamine dyes described in U.S. Pat. Nos. 5,936,087, 5,750,409, 5,366,860, 5,231,191, 5,840,999, 5,847,162, and 6,080,852 (Lee et al.), PCT Publications WO 97/36960 and WO 99/27020, Sauer et al., J. Fluorescence 5(3):247-261 (1995), Arden-Jacob, Neue Lanwellige Xanthen-Farbstoffe für Fluoreszenzsonden und Farbstoff Laser, Verlag Shaker, Germany (1993), and Lee et al., Nucl. Acids Res. 20:2471-2483 (1992). Also included within the definition of “rhodamine-type xanthene ring” are the extended-conjugation xanthene rings of the extended rhodamine dyes described in U.S. application Ser. No. 09/325,243 filed Jun. 3, 1999.

In some embodiments, the dye comprises a fluorescein-type parent xanthene ring having the structure:

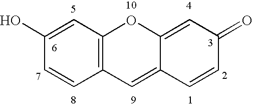

In the fluorescein-type parent xanthene ring depicted above, one or more of the carbons at positions C1, C2, C4, C5, C7, C8 and C9 can be independently substituted with a wide variety of the same or different substituents, as described above for the parent xanthene rings. C9 may be substituted with hydrogen or other substituent, such as an orthocarboxyphenyl or ortho(sulfonic acid)phenyl group. Exemplary fluorescein-type parent xanthene rings include, but are not limited to, the xanthene rings of the fluorescein dyes described in U.S. Pat. Nos. 4,439,356, 4,481,136, 4,933,471 (Lee), U.S. Pat. No. 5,066,580 (Lee), U.S. Pat. Nos. 5,188,934, 5,654,442, and 5,840,999, WO 99/16832, and EP 050684. Also included within the definition of “fluorescein-type parent xanthene ring” are the extended xanthene rings of the fluorescein dyes described in U.S. Pat. Nos. 5,750,409 and 5,066,580.

In some embodiments, the dye comprises a rhodamine dye, which can comprise a rhodamine-type xanthene ring in which the C9 carbon atom is substituted with an orthocarboxy phenyl substituent (pendent phenyl group). Such compounds are also referred to herein as orthocarboxyfluoresceins. In some embodiments, a subset of rhodamine dyes are 4,7,-dichlororhodamines. Typical rhodamine dyes can include, but are not limited to, rhodamine B, 5-carboxyrhodamine, rhodamine X (ROX), 4,7-dichlororhodamine X (dROX), rhodamine 6G (R6G), 4,7-dichlororhodamine 6G, rhodamine 110 (R110), 4,7-dichlororhodamine 110 (dR110), tetramethyl rhodamine (TAMRA) and 4,7-dichloro-tetramethylrhodamine (dTAMRA). Additional rhodamine dyes can be found, for example, in U.S. Pat. No. 5,366,860 (Bergot et al.), U.S. Pat. No. 5,847,162 (Lee et al.), U.S. Pat. No. 6,017,712 (Lee et al.), U.S. Pat. No. 6,025,505 (Lee et al.), U.S. Pat. No. 6,080,852 (Lee et al.), U.S. Pat. No. 5,936,087 (Benson et al.), U.S. Pat. No. 6,111,116 (Benson et al.), U.S. Pat. No. 6,051,719 (Benson et al.), U.S. Pat. Nos. 5,750,409, 5,366,860, 5,231,191, 5,840,999, and 5,847,162, U.S. Pat. No. 6,248,884 (Lam et al.), PCT Publications WO 97/36960 and WO 99/27020, Sauer et al., 1995, J. Fluorescence 5(3):247-261, Arden-Jacob, 1993, Neue Lanwellige Xanthen-Farbstoffe für Fluoresenzsonden und Farbstoff Laser, Verlag Shaker, Germany, and Lee et al., Nucl. Acids Res. 20(10):2471-2483 (1992), Lee et al., Nucl. Acids Res. 25:2816-2822 (1997), and Rosenblum et al., Nucl. Acids Res. 25:4500-4504 (1997), for example. In some embodiments, the dye comprises a 4,7-dichloro-orthocarboxyrhodamine. In some embodiments, the dye comprises a fluorescein dye, which comprises a fluorescein-type xanthene ring in which the C9 carbon atom is substituted with an orthocarboxy phenyl substituent (pendent phenyl group). One typical subset of fluorescein-type dyes are 4,7,-dichlorofluoresceins. Typical fluorescein dyes can include, but are not limited to, 5-carboxyfluorescein (5-FAM), 6-carboxyfluorescein (6-FAM). Additional typical fluorescein dyes can be found, for example, in U.S. Pat. Nos. 5,750,409, 5,066,580, 4,439,356, 4,481,136, 4,933,471 (Lee), U.S. Pat. No. 5,066,580 (Lee), U.S. Pat. No. 5,188,934 (Menchen et al.), U.S. Pat. No. 5,654,442 (Menchen et al.), U.S. Pat. No. 6,008,379 (Benson et al.), and U.S. Pat. No. 5,840,999, PCT publication WO 99/16832, and EPO Publication 050684. In some embodiments, the dye comprises a 4,7-dichloro-orthocarboxyfluorescein. In some embodiments, the dye can be a cyanine, phthalocyanine, squaraine, or bodipy dye, such as described in the following references and references cited therein: U.S. Pat. No. 5,863,727 (Lee et al.), U.S. Pat. No. 5,800,996 (Lee et al.), U.S. Pat. No. 5,945,526 (Lee et al.), U.S. Pat. No. 6,080,868 (Lee et al.), U.S. Pat. No. 5,436,134 (Haugland et al.), U.S. Pat. No. 5,863,753 (Haugland et al.), U.S. Pat. No. 6,005,113 (Wu et al.), and WO 96/04405 (Glazer et al.).

As used herein, the term “adjacent” refers to two oligonucleotides hybridized on a complementary nucleotide sequence in a position such that their 5′ and 3′ termini are abutting and capable of being ligated together. As used herein, the term adjacent shall further include nearly adjacent hybridization of two oligonucleotides in such a fashion that a transient nucleotide gap can be filled in to produce abutting termini capable of being ligated together. Further, the term adjacent shall also include hybridization of oligonucleotides to form flap structures, the cleavage of which allows abutting termini to be ligated together.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “mobility-dependent analytical technique” refers to any means for separating different molecular species based on differential rates of migration of those different molecular species in one or more separation techniques. Exemplary mobility-dependent analysis techniques include gel electrophoresis, capillary electrophoresis, chromatography, capillary electrochromatography, mass spectroscopy, sedimentation, e.g., gradient centrifugation, field-flow fractionation, multi-stage extraction techniques and the like. Descriptions of mobility-dependent analytical techniques can be found in, among other places, U.S. Pat. Nos. 5,470,705, 5,514,543, 5,580,732, 5,624,800, and 5,807,682, PCT Publication No. WO 01/92579, Fu et al., Current Opinion in Biotechnology, 2003, 14:1:96-100, D. R. Baker, Capillary Electrophoresis, Wiley-Interscience (1995), Biochromatography: Theory and Practice, M. A. Vijayalakshmi, ed., Taylor & Francis, London, U.K. (2003); and A. Pingoud et al., Biochemical Methods: A Concise Guide for Students and Researchers, Wiley-VCH Verlag GmbH, Weinheim, Germany (2002).

### Exemplary Embodiments

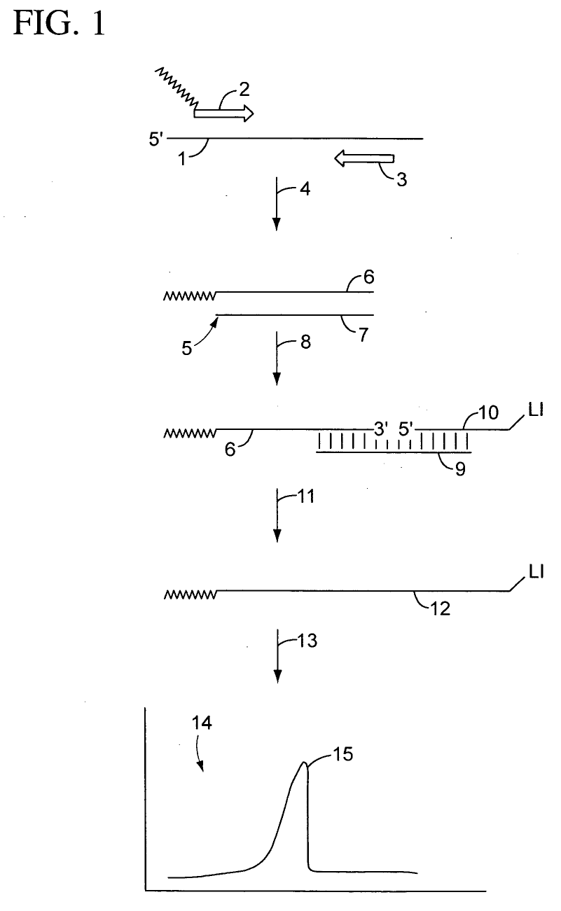

FIG. 1 depicts the formation and detection of a multi-labeled polynucleotide according to some embodiments of the present teachings. Depicted first is amplification of the polynucleotide (1) with a labeled primer (2, here the label is depicted as jagged and can be considered to represent a mobility modifier) and an unlabeled primer (3). After a PCR (4), an amplicon is formed (5) comprising a labeled extension product (6) and an unlabeled extension product (7). Hybridization (8) of a bridge oligonucleotide (9) to the 3′end region of the labeled extension product (6) allows for the hybridization of a labeled tag oligonucleotide (10, here the label is depicted as an L1 and can be considered to represent a florophore) adjacent to the labeled extension product (6). Following ligation (11), a multi-labeled polynucleotide (12) is formed. Analysis (13) via a mobility dependent analysis technique such as capillary electrophoresis can detect the multi-labeled polynucleotide by the peak (15) on the electropherrogram (14), representing its distinct color and mobility.

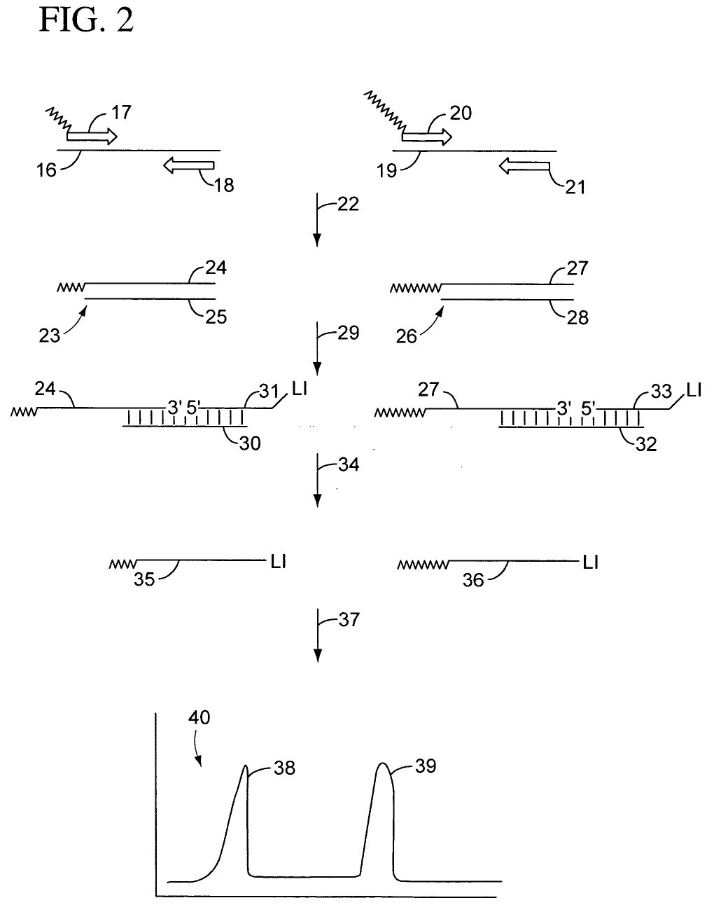

FIG. 2 depicts the formation and detection of two multi-labeled polynucleotides according to some embodiments of the present teachings. Depicted first is the amplification of a first polynucleotide (16) with a first labeled primer (17, here the label is depicted as jagged and can be considered to represent a first mobility modifier) and a first unlabeled primer (18), along with a second polynucleotide (19) with a second labeled primer (20, here the label is depicted as jagged and can be considered to represent a second mobility modifier) and a second unlabeled primer (21). After the PCR (22), a first amplicon is formed (23) and a second amplicon is formed (26). The first amplicon (23) comprises a first labeled extension product (24) and a first unlabeled extension product (25). The second amplicon (26) comprises a second labeled extension product (27) and a second unlabeled extension product (28). Hybridization (29) of a first bridge oligonucleotide (30) to the 3′end region of the first labeled extension product (24) allows for the hybridization of a first labeled tag oligonucleotide (31, here the label is depicted as an L1 and can be considered to represent a florophore) adjacent to the first labeled extension product (24). Hybridization (29) of a second bridge oligonucleotide (32) to the 3′ end region of the second labeled extension product (27) allows for the hybridization of a second labeled tag oligonucleotide (33, here the label is depicted as an L1 and can be considered to represent a florophore) adjacent to the second labeled extension product (27). Following ligation (34), a first multi-labeled polynucleotide (35) and a second multi-labeled polynucleotide (36) is formed. Analysis (37) via a mobility dependent analysis technique such as capillary electrophoresis provides an electropherrogram (40) that can indicate the first multi-labeled polynucleotide by the first peak (39) and the second multi-labeled polynucleotide by the second peak (38) based on the florophore present in each multi-labeled polynucleotide, and the distinct mobility modifier encoded in the PCR.

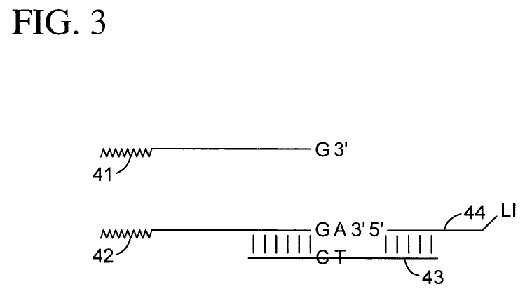

FIG. 3 depicts one aspect of the present teachings wherein those labeled extension products in amplicons resulting from a PCR that comprise a template-independent adenine addition can be selectively queried in a ligation reaction comprising a bridge oligonucleotide that comprises a corresponding complementary thymine. Here, a first labeled extension product comprising a template independent adenine (42, shown with the 3′ located nucleotides GA) is present in an amplification reaction product mixture comprising a first labeled extension product that does not contain a template independent adenine (41, shown with the 3′ G, indicating the absence of template independent A addition). Providing a bridge oligonucleotide (43) that comprises the appropriate corresponding complementary thymine results in the selective ligation of a labeled tag oligonucleotide (44) to only those labeled extension products that comprise the template independent adenine.

It will be appreciated that the present teachings further contemplate a variety of approaches for making labeled polynucleotides. In some embodiments, an unlabeled extension product can be ligated to a labeled tag oligonucleotide form a labeled polynucleotide. In some embodiments, a labeled extension product can be ligated to a labeled tag oligonucleotide to form a multi-labeled polynucleotide. In some embodiments, a labeled or unlabeled extension product can be ligated to a labeled tag oligonucleotide, wherein the labeled tag oligonucleotide comprises at least two different labels, such as both a mobility modifier and a florophore.

The present teachings can be employed in the context of a multiplexed PCR amplification of a plurality of polymorphic microsatellite loci, as for example in a forensics HID setting, a paternity setting, livestock tracking setting, and other appropriate contexts. In such a multiplexed reaction, a plurality of microsatellites can be PCR amplified with a plurality of primer pairs, wherein the first primer in each primer pair can be a labeled primer, and the second primer in each primer pair can be an unlabeled primer. For example, the first primer of each primer pair corresponding to a micosatellite can comprise a distinct mobility modifier. The length of the resulting amplicons, along with the size information conferred by the mobility modifier, can be used to identify the amplicon on a mobility dependent analysis technique such as capillary electrophoresis. In such a scenario, at least one labeled tag oligonucleotide can be ligated to the plurality of amplicons by employing at least one bridge oligonucleotide, wherein the plurality of labeled tag oligonucleotides subsequently comprise a label such as a florophore to allow for the visualization of the resulting multi-labeled polynucleotides.

The present teachings contemplate a plurality of scenarios in which at least one labeled tag oligonucleotide can be ligated to a plurality of amplicons using at least one bridge oligonucleotide. As a first non-limiting illustration, one can envision a context in which a plurality of microsatellites is amplified with a plurality of primer pairs, wherein each primer pair comprises a labeled primer comprising a distinct mobility modifier. A plurality of different bridge oligonucleotides can then be employed, each one of which can comprise a region complementary to the 3′ end of each of the plurality of amplicons. The plurality of bridge oligonucleotides can each further comprise a region complementary with one of a plurality of labeled tag oligonucleotides, wherein each labeled tag oligonucleotide comprises a florophore. In such a scenario, each of the plurality of amplified loci can be ligated with a specific labeled tag oligonucleotide, resulting in each amplified locus bearing a distinct mobility modifer and a label. Analysis of the resulting multi-labeled polynucleotides on a mobility dependent analysis technique such as capillary electrophoresis results in a signature of peaks on an electropherrogram indicative of a given sample. Further, heterozygosity and homozygosity of the amplified loci can also be inferred from the peaks, due to length variation in the amplified loci resulting from varying number of tandem repeats. Thus, N target microsatellites can be amplified with N primer pairs with N mobility modifiers, and N bridge oligonucleotides employed along with N labeled tag oligonucleotides. Any heterozygosity in the sample can result in a number of peaks in an electropherrogram in excess of N. Of course, an additional level of information can be employed in such a scenario by employing different florophores on different labeled tag oligonucleotides, thereby allowing for new channels of color information. In some embodiments of this scenario, mobility modifiers need not be included in the primer pair in the amplification, and the size of the amplicons themselves, along with the ligated labeled tag oligonucleotide, used to produce a signature of peaks on an electropherrogram indicative of a given sample.

As a second non-limiting illustration, one can envision a context in which a plurality of microsatellites is amplified with a plurality of primer pairs, wherein each primer pair comprises a labeled primer comprising a distinct mobility modifier. A plurality of bridge oligonucleotides can then be employed, each bridge comprising a region complementary to the 3′ end of each of the plurality of amplicons. The plurality of bridge oligonucleotides can further comprise a region complementary with a single labeled tag oligonucleotide, wherein the labeled tag oligonucleotide comprises a florophore. Analysis of the resulting multi-labeled polynucleotides on a mobility dependent analysis technique such as capillary electrophoresis results in a signature of peaks on an electropherrogram indicative of a given sample. Further, heterozygosity and homozygosity of the amplified loci can also be inferred from the peaks, due to length variation in the amplified loci resulting from varying number of tandem repeats. Thus, N target microsatellites can be amplified with N primer pairs with N mobility modifiers, and N bridge oligonucleotides employed along with a single labeled tag oligonucleotide. Any heterozygosity in the sample can result in a number of peaks in an electropherrogram in excess of N. In some embodiments of this scenario, mobility modifiers need not be included in the primer pair in the amplification, and the size of the amplicons themselves, along with the ligated labeled tag oligonucleotide, used to produce a signature of peaks on an electropherrogram indicative of a given sample.

As a third non-limiting illustration, one can envision a context in which a plurality of microsatellites is amplified with a plurality of primer pairs, wherein each primer pair comprises a labeled primer comprising a distinct mobility modifier. Further, the amplification comprises an unlabeled primer, wherein the unlabeled primer comprises a universal identifying portion on its 5′ end. A single bridge oligonucleotide can then be employed, with the bridge comprising a region complementary to the universal identifying portion incorporated into the plurality of amplicons. The single bridge oligonucleotide can further comprise a region complementary with a single labeled tag oligonucleotide, wherein the labeled tag oligonucleotide comprises a florophore. Analysis of the resulting multi-labeled polynucleotides on a mobility dependent analysis technique such as capillary electrophoresis results in a signature of peaks on an electropherrogram indicative of a given sample. Further, heterozygosity and homozygosity of the amplified loci can also be inferred from the peaks, due to length variation in the amplified loci resulting from varying number of tandem repeats. Thus, N target microsatellites can be amplified with N primer pairs with N mobility modifiers, and a single bridge oligonucleotides employed along with a single labeled tag oligonucleotide. Any heterogzygosity in the sample can result in a number of peaks in an electropherrogram in excess of N. In some embodiments of this scenario, mobility modifiers need not be included in the primer pair in the amplification, and the size of the amplicons themselves, along with the ligated labeled tag oligonucleotide, used to produce a signature of peaks on an electropherrogram indicative of a given sample. It will be appreciated that aspects of the first illustration supra, the second illustration supra, and the third illustration supra can be employed in various combinations.

The present teachings contemplate a plurality of scenarios in which ligation of the labeled tag oligonucleotides can be performed on an amplicon. In some embodiments, bridge oligonucleotides and labeled tag oligonucleotides can be present in stoichiometric excess relative to the amplicon concentration, thus increasing the prevalence of hybridization and ligation. In some embodiments, an asymmetric PCR can be performed to bias the generation of a single stranded amplicon suitable for hybridization and ligation of the bridge oligonucletide and labeled tag oligonucleotides. In some embodiments, an asychronous PCR can be performed to bias the generation of a single stranded amplicon suitable for hybridization and ligation of the bridge oligonucletide and labeled tag oligonucleotides. In some embodiments, the primers in an amplification reaction can be designed in such fashion as to incorporate a recognition sequence for a restriction endonuclease, such that treatment of the resulting amplicons can result in cleavage of the primer sequence and the resultant formation of a single-stranded overhang. Such an overhang can facilitate the hybridization and ligation of the bridge oligonucleotide and labeled tag oligonucleotide. In some embodiments, the bridge oligonucleotide can comprise a blocking moiety at its 3′ end to prevent unwanted extension. In some embodiments, a minor groove binder (MGB) can be included on the 3′ end of the bridge oligonucleotide to provide increased thermal stability. In some embodiments, blocking moieties such as an MGB, polyethylene glycol (PEG), C18, and/or tetra methoxy uracil can be employed on the bridge oligonucleotide. In some embodiments, an affinity moiety such as biotin can be included in one of the PCR primers to allow for immobilization of the amplicons. In some embodiments employing such an affinity moiety, the immobilized stranded can be the strand to which ligation of the labeled tag oligonucleotide occurs. In some embodiments employing an affinity moiety, the strand eluted from the immobilized strand can be the strand to which ligation of the labeled tag oligonucleotide occurs.

The present teachings further contemplate embodiments in which a strand of an amplicon and an oligonucleotide hybridize on a bridge oligonucleotide, wherein a nucleotide extension reaction is performed with labeled nucleotides to extend the olignucleotide and thereby form a labeled tag oligonucleotide. In some embodiments, a single base extension reaction comprising florophore-labeled dideoxynucleotides can be performed. In some embodiments, and extension reaction can be performed using labeled deoxynucleotides.

Detection and Quantification

Detection and quantification can be carried out using a variety of procedures, including for example mobility dependent analysis techniques (for example capillary or gel electrophoresis), solid support comprising array capture oligonucleotides, various bead approaches (see for example Published P.C.T. Application WO US02/37499), including fiber optics, as well as flow cytometry (for example, FACS).

The use of capillary and gel electrophoresis for detection and quantification of target polynucleotides is well known, see for example, Grossman, et. al., “High-density Multiplex Detection of Nucleic Acid Sequences: Oligonucleotide Ligation Assay and Sequence-coded Separation,” Nucl. Acids Res. 22(21): 4527-34 (1994), Slater et al., Current Opinion in Biotechnology, 2003, 14:1:58-64, product literature for the Applied Biosystems 3100, 3700, and 3730 capillary electrophoresis instruments, and product literature for the SNPlex Genotyping System Chemistry Guide, also from Applied Biosystems.

Additional mobility dependent analysis techniques that can provide for detection and quantification according to the present teachings include mass spectroscopy (optionally comprising a deconvolution step via chromatography), collision-induced dissociation (CID) fragmentation analysis, fast atomic bombardment and plasma desorption, and electrospray/ionspray (ES) and matrix-assisted laser deorption/ionization (MALDI) mass spectrometry. In some embodiments, MALDI mass spectrometry can be used with a time-of-flight (TOF) configuration (MALDI-TOF, see for example Published P.C.T. Application WO 97/33000), and MALDI-TOF-TOF (see for example Applied Biosystems 4700 Proteomics Discovery System product literature). Additional mass spectrometry approaches for detection and quantification are described for example in the Applied Biosystems Qtrap LC/MS/MS System product literature, the Applied Biosystems QSTAR XL Hybrid LC/MS/MS System product literature, the Applied Biosystems Q TRAP™ LC/MS/MS System product literature, and the Applied Biosystems Voyager-DE™ PRO Biospectrometry Workstation product literature.

The use of a solid support with an array of capture oligonucleotides is fully disclosed among other places in pending provisional U.S. Non-Provisional application Ser. No. 10/854,482 to Barany et al.,. In some embodiments when using such arrays, the oligonucleotide primers or probes used in the herein-described PCR and/or LDR phases, respectively, can have an addressable hybridization tag (for example, an identifying portion). After the LDR or PCR phases are completed, the addressable hybridization tags of the products of such processes remain single stranded and are caused to hybridize to the capture oligonucleotides during a capture phase. See for example, C. Newton, et al., “The Production of PCR Products With 5′ Single-Stranded Tails Using Primers That Incorporate Novel Phosphoramidite Intermediates,” Nucl. Acids Res. 21(5):1155-62 (1993), Carrino Published P.C.T. Application WO 096152371A1. The present teachings further contemplate a variety of additional array-based procedures known in the art, including but not limited to dot-blots (see for example Andersen and Young, in Nucleic Acid Hybridization-A Practical Approach, IRL Press, Chapter 4, pp. 73-111, 1985, and EPA 0228075, and for the detection of overlapping dines and the construction of genomic maps Evans, G. A. U.S. Pat. No. 5,219,726), reverse dot blots, and matrix hybridization (see Beattie et al., in The 1992 San Diego Conference: Genetic Recognition, November, 1992), photolithographically generated arrays (see for example Fodor et al., 1991, Science, 251: 767-777. as well as Geneflex Tag Arrays from Affymetrix), universal arrays as described for example in Published P.C.T. application WO 9731256A2, WO 0179548A2, WO 0056927A3, product literature associated with commercially available spotted arrays from Agilent, product literature associated with the commercially available Applied Biosystems Expression Array System, printing-based arrays commercially available from Hewlett Packard and Rosetta-Merck, electrode arrays, three dimensional “gel pad” arrays, as well as three-dimensional array methods such as FACS. In some embodiments, detection and quantification can be carried out on a variety of bead-based formats, described for example in Published P.C.T. Applications US98/21193, US99/14387, US98/05025, WO 98/50782, U.S. Ser. Nos. 09/287,573, 09/151,877, 09/256,943, 09/316,154, 60/119,323, and 09/315,584. Also see “Microsphere Detection Guide” from Gangs Laboratories, Fishers Ind. for a discussion of beads and microspheres. In some embodiments, detection and quantification can be carried out with a fiber bundle or array, as is generally described in U.S. Ser. Nos. 08/944,850 and 08/519,062, PCT US 98/05025, and PCT US 98/09163, as well as U.S. Ser. No. 09/473,904.

In some embodiments of the present teachings, detection can achieved by various real-time PCR approaches. Devices comprising a thermal cycler, light beam emitter, and a fluorescent signal detector, have been described, e.g., in U.S. Pat. Nos. 5,928,907; 6,015,674; and 6,174,670, and include, but are not limited to the ABI Prism® 7700 Sequence Detection System (Applied Biosystems, Foster City, Calif.), the ABI GeneAmp® 5700 Sequence Detection System (Applied Biosystems, Foster City, Calif.), the ABI GeneAmp® 7300 Sequence Detection System (Applied Biosystems, Foster City, Calif.), and the ABI GeneAmp® 7500 Sequence Detection System (Applied Biosystems, Foster City, Calif.).

### Additional Embodiments

Some embodiments of the present teachings provide a step of amplifying, a step of ligating, a step of detecting, or combinations thereof.

Kits

In certain embodiments, the present teachings also provide kits designed to expedite performing certain methods. In some embodiments, kits serve to expedite the performance of the methods of interest by assembling two or more components used in carrying out the methods. In some embodiments, kits may contain components in pre-measured unit amounts to minimize the need for measurements by end-users. In some embodiments, kits may include instructions for performing one or more methods of the present teachings. In certain embodiments, the kit components are optimized to operate in conjunction with one another.

Some embodiments of the present teachings provide a means for amplifying, a means for ligating, and a means for detecting, or combinations thereof.

Some embodiments of the present teachings contemplate a kit comprising a primer pair, a bridge oligonucleotide, and a labeled tag oligonucleotide, wherein one primer in the primer pair comprises a label. Some embodiments further contemplate a plurality of primer pairs capable of selectively amplifying a plurality of microsatellites, at least one bridge oligonucleotide, and at least one labeled tag oligonucleotide, wherein one primer in each primer pair of the plurality of primer pairs comprises a label. In some embodiments of the present teachings, one primer in each primer pair of the plurality of primer pairs comprises a label, wherein the label comprises a florophore. Additional kit configurations are contemplated by the present teachings, as will be appreciated by one of ordinary skill in the art after reading the entirety of this application.

While the present teachings have been described in terms of these exemplary embodiments, the skilled artisan will readily understand that numerous variations and modifications of these exemplary embodiments are possible without undue experimentation. All such variations and modifications are within the scope of the current teachings. Aspects of the present teachings may be further understood in light of the following example, which should not be construed as limiting the scope of the teachings in any way.

### EXAMPLE 1

The AmpFλSTR® Identifiler™ PCR Amplification kit (Applied Biosystems P/N 4322288) contains primers to amplify 15 STR loci plus the sex determining locus Amelogenin. This kit was used to demonstrate feasibility of the ligation labeling approach of the present teachings. Bridge oligonucleotides were designed for four of the STR loci in this kit.

First, a multiplex PCR reaction was performed with the Identifiler™ PCR Amplification kit using 1 ng of male control DNA (a no template control was also included). The reactions were amplified according to manufacture's protocol but an additional step was added at the end to heat-kill the AmpliTaq Gold polymerase (heating at 99.5 C for 30 min).

Next, ligation reactions were set up by combining the following amounts of components: 5 μl OLA Buffer (Applied Biosystems SNPlex System OLA Master Mix), 3 μl bridge oligonucleotides and labeled tag oligonucleotides with mobility modifiers (Amelogenin, D3, THO1 and vWA bridge oligonucleotides, and mobility modifier-conjugated labeled tag oligonucleotides were each at 250 nM in solution), and 2 μl Identifiler amplified reaction sample DNA or No Template Control (NTC))

Next, reaction plates were placed in a 9700 and the following thermal cycle program performed: 48 C for 5 min, followed by 90 C for 20 min, followed by 5×[94 C/15 sec, 60 C/30 sec, 51 C/30 sec], followed by a 4 C hold. A reduced ramp rate (2%) for the annealing step was used (between the 60 C/30 second step and the 51 C/30 second step.

After thermal cycling, samples were prepared for 3100 electrophoresis with 1.5 μl of ligation reaction, 8.7 μl Hi-Di Formamidem and 0.3 μl GS500-LIZ size standard.

Next, a GeneScan run was performed on an Applied Biosystems 3100 with 36 cm capillaries and POP-4 polymer. The Control system setup comprised using short (˜25 nucleotide) dye-labeled synthetic targets that matched the 3′ ends of the Identifiler amplified loci for amelogenin (PET), D3 (VIC), THO1 (VIC) and vWA (NED). The synthetic targets were designed in two configurations: a “plus-A” version that contained a 3′ terminal “A” nucleotide to simulate normal non-template “A” addition by the STR amplification system, and a “minus-A” version that lacked the terminal “A” and represented an incomplete plus A addition, as sometimes occurs. This was done as a positive control for the ligation tagging reactions, and also as a test of the system's specificity. The specificity can be determined by the bridge oligonucleotides, which were designed to allow the ligation of only the “plus-A” amplified STR products. Since the synthetic targets are dye labeled, they would be predicted to appear in the “read region” of the electropherogram only when successful ligation occurs.

The labeled synthetic targets were substituted for Identifiler amplified samples in control ligation reactions. A multiplex solution containing all four “plus-A” or “minus-A” synthetic targets (Amel, D3, THO1 and vWA), each at 25 nM, was added to ligation reactions and thermal cycled as described above.

All of the foregoing cited references are expressly incorporated by reference. Recognizing the difficulty of ipsissima verba in multiple documents related to the complex technology of molecular biology, it will be appreciated that when deviances in the nature of a definition are encountered, the definitions provided in the instant application will control.

We claim:

1. A method of forming a multi-labeled polynucleotide comprising;
hybridizing a labeled primer to a target polynucleotide;
extending the labeled primer to form a labeled extension product; and,
ligating a labeled tag oligonucleotide to the labeled extension product to form a multi-labeled polynucleotide.

2. A method of forming a multi-labeled polynucleotide comprising;
providing a labeled primer and an unlabeled primer;
amplifying a target polynucleotide with the labeled primer and the unlabeled primer in a PCR to form an amplicon, wherein the amplicon comprises a labeled extension product and an unlabeled extension product;
ligating a labeled tag oligonucleotide to the labeled extension product to form a multi-labeled polynucleotide.

3. The method according to claim 2 wherein the multi-labeled polynucleotide is an amplified microsatellite.

4. The method according to claim 2 wherein the ligation comprises a bridge oligonucleotide, wherein the labeled tag oligonucleotide and the labeled extension product hybridize adjacent to one another on the bridge oligonucleotide prior to ligation.

5. The method according to claim 4 wherein the bridge oligonucleotide comprises a thymine that forms a complementary base-pair with a template-independent adenine at the 3′ terminal end of the labeled extension product.

6. The method according to claim 2 wherein the labeled primer comprises a mobility modifier and the labeled tag oligonucleotide comprises a florophore.

7. The method according to claim 5 wherein the florophore is selected from the group comprising 6FAM, VIC, NED, PET, JOE, 5FAM, TET, ROX, HEX, and TAMARA.

8. The method according to claim 2 wherein the labeled primer comprises a florophore and the unlabeled tag oligonucleotide comprises a mobility modifier.

9. A method of forming at least two different multi-labeled polynucleotides comprising;
providing a first primer pair specific for a first target polynucleotide, wherein the first primer pair comprises a first labeled primer and a first unlabeled primer;
providing a second primer pair specific for a second target polynucleotide, wherein the second primer pair comprises a second labeled primer and a second unlabeled primer;
amplifying the first target polynucleotide and the second target polynucleotide in a PCR to form a first amplicon and a second amplicon, wherein the first amplicon comprises a first labeled extension product and a first unlabeled extension product, and the second amplicon comprises a second labeled extension product and a second unlabeled extension product;
ligating a first labeled tag oligonucleotide to the first labeled extension product ligating a second labeled tag oligonucleotide to the second labeled extension product;
wherein the ligating comprises a first bridge oligonucleotide and a second bridge oligonucleotide, wherein the first labeled tag oligonucleotide and the first labeled extension product hybridize adjacent to one another on the first bridge oligonucleotide, and wherein the second labeled tag oligonucleotide and the second labeled extension product hybridize adjacent to one another on the second bridge oligonucleotide; and
forming at least two different multi-labeled polynucleotides.

10. The method according to claim 9 wherein the first multi-labeled polynucleotide is a first amplified microsatellite and the second multi-labeled polynucleotide is a second amplified microsatellite.

11. The method according to claim 10 wherein the first amplified microsatellite and the second amplified microsatellite are from a sample comprising human remains.

12. The method according to claim 9 wherein the first bridge oligonucleotide comprises a thymine that forms a complementary base-pair with a template-independent adenine at the 3′ terminal end of the first labeled extension product, the second bridge oligonucleotide comprises a thymine that forms a complementary base-pair with a template-independent adenine at the 3′ terminal end of the second labeled extension product, or a first bridge oligonucleotide comprises a thymine that forms a complementary base-pair with a template-independent adenine at the 3′ terminal end of the first labeled extension product and a second bridge olignucleotide comprises a thymine that forms a complementary base-pair with a template-independent adenine at the 3′ terminal end of the second labeled extension product.

13. The method according to claim 9 wherein the first labeled primer comprises a mobility modifier and the first labeled tag oligonucleotide comprises a florophore, and the second labeled primer comprises a mobility modifier and the second labeled tag oligonucleotide comprises a florophore.

14. The method according to claim 13 wherein the mobility modifier of the first labeled primer differs from the mobility modifier of the second labeled primer.

15. The method according to claim 13 wherein the florophore of the first labeled tag oligonucleotide differs from the florophore of the second labeled tag oligonucleotide.

16. The method according to claim 13 wherein the florophore is at least one of 6FAM, VIC, NED, PET, JOE, 5FAM, TET, ROX, HEX, and TAMARA.

17. The method according to claim 9 further comprising providing a plurality of target polynucleotides, forming a plurality of amplicons, and forming a plurality of multi-labeled polynucleotides, wherein the plurality of amplicons comprises at least one of human CSF1 PO, D3S1358, D5S818, D7S820, D8S1179, D13S317, D16 S539, D18S51, D21 S11, FGA, TH01, TPOX, vWA, D2S1338, D19S433, Amelogenin, SE33, DYS19, DYS385a/b, DYS389I/II, DYS390, DYS391, DYS392, DYS393, DYS438, DYS439, DYS437, DYS448, DYS456, DYS458, Y GATA C4 (DYS635), Y GATA H4, or combinations thereof.

18. A kit comprising a primer pair, a bridge oligonucleotide, and a labeled tag oligonucleotide, wherein one primer in the primer pair comprises a label.

19. The kit according to claim 18 further comprising a plurality of primer pairs for specifically amplifying a plurality of microsatellites, at least one bridge oligonucleotide, and at least one labeled tag oligonucleotide, wherein one primer in each primer pair of the plurality of primer pairs comprises a label.

20. The kit according to claim 19 wherein the one primer in each primer pair of the plurality of primer pairs comprises a label, wherein the label comprises a florophore.
